(19) United States
(12) Patent Application Publication
(10) Pub. No.: US 2012/0149657 A1
(43) Pub. Date: Jun. 14, 2012

(54) 2'-FLUORO ARABINO NUCLEOSIDES AND USE THEREOF

(75) Inventors: John A. Secrist, III, Birmingham, AL (US); Anita T. Fowler, Birmingham, AL (US); Kamal N. Tiwari, Birminggham, AL (US)

(73) Assignee: Southern Research Institute, Birmingham (AL)

(21) Appl. No.: 13/320,492

(22) PCT Filed: May 12, 2010
(86) PCT No.: PCT/US10/34463

§ 371 Date: Feb. 29, 2012

Related U.S. Application Data
(60) Provisional application No. 61/177,436, filed on May 12, 2009.

(51) Int. Cl. A61K 31/706; A61P 35/00; A61K 31/7076; C07H 19/073; A61K 31/7068

(52) U.S. Cl. 514/46; 536/28.5; 514/49; 514/43

(57) ABSTRACT

A method of treating cancer using certain 2′-fluoro arabino nucleosides is provided. Also provided are compounds represented by the formula: (I & A) wherein R is alkyl; and pharmaceutically acceptable salts thereof; and pharmaceutical compositions containing these compounds.

### FEDERALLY SPONSORED RESEARCH AND DEVELOPMENT

This invention was partially supported by a NIH Grant No. CA34200 from National Institute of Health and the US Government has certain rights in the invention.

### TECHNICAL FIELD

The present disclosure relates to certain 2′-fluoro arabino nucleosides. The present disclosure also relates to pharmaceutical compositions comprising the disclosed compounds. The present invention is also concerned with treating patients suffering from cancer by administering to the patients certain 2′-fluoro arabino nucleosides compounds. Compounds employed according to the present invention have exhibited good anticancer activity. The present disclosure also relates to a method for producing the disclosed compounds.

### BACKGROUND OF DISCLOSURE

A considerable amount of research has occurred over the years related to developing treatments against cancers to inhibit and kill tumor cells. Some of this research has resulted in achieving some success in finding clinically approved treatments. Nevertheless, efforts continue at an ever-increasing rate in view of the extreme difficulty in uncovering promising anticancer treatments. For example, even when a compound is found to have cytotoxic activity, there is no predictability of it being selective against cancer cells.

Even though significant advances have occurred in the treatment of cancer, it still remains a major health concern. Cancer has been reported as the leading cause of death in the United States with one of every four Americans likely to be diagnosed with the disease.

Notwithstanding the advances in treatments for cancer and other diseases there still remains room for improved drugs that are effective for the desired treatment, while at the same time exhibiting reduced adverse side effects.

### SUMMARY OF DISCLOSURE

The present disclosure relates compounds represented by the formula:

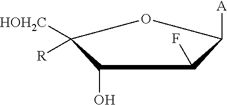

wherein A is

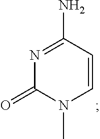

and
wherein R is alkyl; and pharmaceutically acceptable salts thereof.

Another aspect of the present disclosure relates to pharmaceutical compositions containing the above-disclosed compounds.

Also disclosed is a method of treating cancer in a mammal comprising administering to the mammal an effective treatment amount of a compound represented by the formula:

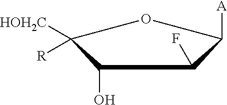

wherein R is alkyl,
wherein A is selected from the group consisting of

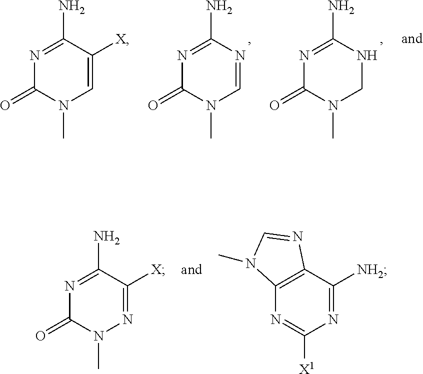

and

wherein X is selected from the group consisting of hydrogen, halo, alkoxy, alkyl, haloalkyl, alkenyl, haloalkenyl, alkynyl, amino, monoalkylamino, dialkylamino, cyano and nitro; and X¹ is selected from the group consisting of hydrogen, halo, alkyl, alkenyl, alkynyl, amino, monoalkylamino, and dialkylamino; and pharmaceutically acceptable salts thereof.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF DRAWING

FIG. 1 illustrates the effect of compound according to the present disclosure on CAKI-1 tumor growth.

### BEST AND VARIOUS MODES

The present disclosure relates compounds represented by the formula:

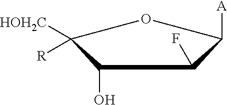

wherein A is

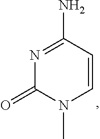

and
wherein R is alkyl; and pharmaceutically acceptable salts thereof.

The present disclosure also relates to a method of treating cancer in a mammal comprising administering to the mammal an effective treatment amount of a compound represented by the formula:

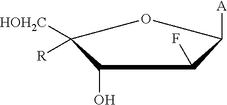

wherein R is alkyl,
wherein A is selected from the group consisting of

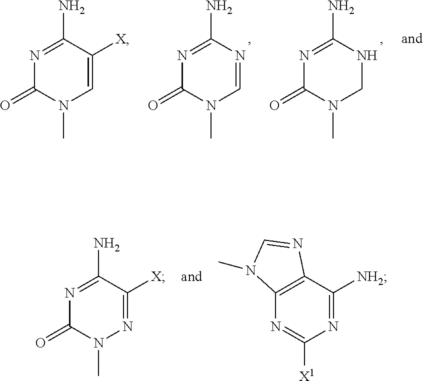

and

wherein X is selected from the group consisting of hydrogen, halo, alkoxy, alkyl, haloalkyl, alkenyl, haloalkenyl, alkynyl, amino, monoalkylamino, dialkylamino, cyano and nitro; and X¹ is selected from the group consisting of hydrogen, halo, alkyl, alkenyl, alkynyl, amino, monoalkylamino, and dialkylamino; and pharmaceutically acceptable salts thereof.

The alkyl groups for R typically contain 1-4 carbon atoms and include methyl, ethyl, i-propyl, n-propyl, i-butyl and n-butyl. The alkyl group can be straight or branched chain. The preferred alkyl group for R is methyl. Examples of halo groups for R are chloro, bromo and preferably fluoro.

Suitable monoalkylamino groups for X contain 1-6 carbon atoms and include monomethylamino, monoethylamino, mono-isopropylamino, mono-n-propylamino, mono-isobutyl-amino, mono-n-butylamino and mono-n-hexylamino. The alkyl moiety can be straight or branched chain.

Suitable dialkylamino groups for Y and X contain 1-6 carbon atoms in each alkyl group. The alkyl groups can be the same or different and can be straight or branched chain. Examples of some suitable groups are dimethylamino, diethylamino, ethylmethylamino, dipropylamino, dibutylamino, dipentylamino, dihexylamino, methylpentylamino, ethylpropylamino and ethylhexylamino.

Suitable halogen groups for X include Cl, Br and F.

Suitable alkyl groups for X typically contain 1-6 carbon atoms and can be straight or branched chain. Some examples are methyl, ethyl, i-propyl, n-propyl, i-butyl, n-butyl, pentyl and hexyl.

Suitable haloalkyl groups typically contain 1-6 carbon atoms and can be straight or branched chain and include Cl, Br or F substituted alkyl groups including the above specifically disclosed alkyl groups.

Suitable alkoxy groups typically contain 1-6 carbon atoms and include methoxy, ethoxy, propoxy and butoxy.

Suitable alkenyl groups typically contain 2-6 carbon atoms and include ethenyl and propenyl.

Suitable haloalkenyl groups typically contain 1-6 carbon atoms and include Cl, Br or F substituted alkenyl groups including the above specifically disclosed alkenyl groups.

Suitable alkynyl groups typically contain 1-6 carbon atoms and include ethynyl and propynyl.

Pharmaceutically acceptable salts of the compounds of the present disclosure include those derived from pharmaceutically acceptable inorganic or organic acids. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include alkali such as sodium and ammonia.

The preferred compounds according to the present disclosure are 1-(2-Deoxy-2-fluoro-4-C-methyl-β-D-arabinofuranosyl)cytosine and 1-(2-Deoxy-2-fluoro-4-C-cyano-β-D-arabinofuranosyl)cytosine and most preferably 1-(2-Deoxy-2-fluoro-4-C-methyl-β-D-arabinofuranosyl)cytosine.

Compounds according to the present disclosure can be prepared as discussed below and shown in Scheme 1. The synthesis of 4′-C-hydroxymethyl-2′-fluoro-arabinofuranoside 1 and the corresponding nucleosides^{[1]} have already been reported in the literature. Selective protection of 1 with the monomethoxytrityl (MMT) group was carried out using MMT chloride in pyridine in 30% yield.^{[3]} The undesired isomer 2b and the unreacted 1 were recycled to increase the yield. The selectively blocked intermediate 2a was benzoylated to give 3 in 92% yield, which was then detritylated to afford the sugar intermediate 4 in 89% yield. This 4′-C-hydroxymethyl analogue 4 was converted into 4′-C-phenoxythiocarbonyloxymethyl derivative 5 in 90% yield using phenyl chlorothionoformate. Compound 5 was deoxygenated using 1,1′-azobis(cyclohexane-carbonitrile) (ACCN) and tris(trimethyl)silane to provide 4′-C-methyl analogue 6 in 84% yield.^{[4]} Acetolysis of compound 6 using traditional methods failed to give 1-O-acetyl sugar 8, resulting in either no reaction or gradual decomposition. The methyl glycoside 6 was instead hydrolyzed using 9:1 trifluoroacetic acid/water to provide the hydroxy sugar 7 in 83% yield, which was acetylated to produce compound 8 in 91% yield. This sugar intermediate was converted cleanly into glycosyl bromide 9 using 33% HBr in acetic acid. Attempted conversion of 7 directly to 9 resulted in a complex mixture and a very low yield of 9. The bromosugar 9 was highly reactive and was used directly without purification for coupling reactions.

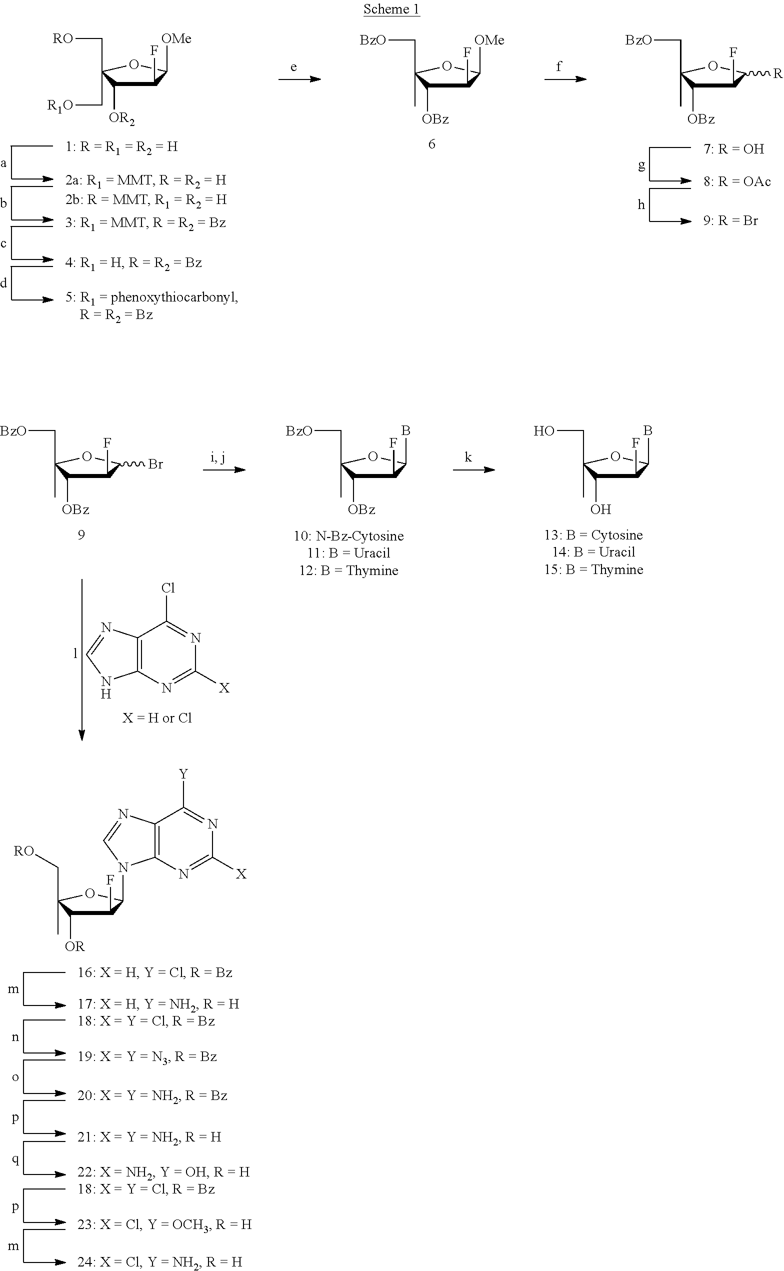

Conditions: (a) MMTr-Cl, pyridine, room temperature, overnight; (b) BzCl, pyridine, room temperature, overnight; (c) 80% AcOH, room temperature, overnight; (d) PhOC(═S)Cl, DMAP, MeCN, room temperature, 3 hours; (e) (TMS)₃SiH, ACCN, toluene, 100° C., 5 hours; (f) TFA/H₂O, 65° C., 24 hours; (g) Ac₂O/pyridine, room temperature, overnight; (h) HBr/AcOH, 5° C. overnight; (i) Bases, BSA, MeCN, room temperature, 1-2 hours; (j) persilylated bases, compound 9, ClCH₂CH₂Cl, 100° C., 4 hours; (k) 0.5N NaOCH₃, MeOH, room temperature, 2-7 hours; (l) NaH, MeCN, room temperature, 6 hours; (m) EtOH, NH₃, 80° C., 16 hours; (n) NaN₃, EtOH, reflux, ½ hours; (o) 10% Pd/C, H₂, 1 atm, EtOH/DMAC, 18 hours; (p) NaOCH₃/MeOH, room temperature, 3 hours; (q) Adenosine deaminase

Coupling of bromosugar 9 with silylated N⁴-benzoylcytosine in situ with BSA gave cytosine nucleosides 10/10α in 54% yield.^{[5,6]} Separation of α, β anomers afforded pure β anomer 10 as the major product (48%) and α anomer 10α as the minor product (6%). Similarly uracil and thymine were coupled with bromo sugar 9 to obtain corresponding nucleosides 11/11α and 12/12α in 71% and 68% yields respectively with the p anomer as the predominant product. Both anomers of cytosine nucleoside 10 were deblocked using sodium methoxide to get the target compounds 13 and 13α. After purification the β anomer 13 was isolated as a hydrochloride salt in 89% yield, and the α anomer 13α was isolated as the free base in 77% yield. In the case of nucleosides 11 and 12, only the β anomers were deblocked using the same procedure to obtain compounds 14 (77%) and 15 (92%) respectively. The α anomers of compounds 11 and 12 were not further utilized and were isolated only for characterization purposes to compare with the β anomers.

A series of purine nucleoside analogues were prepared through the coupling of bromosugar 9 with 6-chloropurine and with 2,6-dichloropurine. Sodium salt coupling of 9 with 6-chloropurine gave the desired β nucleoside 16 (36%) and α anomer 16α (14%).^{[7]} Separate treatment with ethanolic ammonia gave the target compound 17 (74%) and α anomer 17α (49%), respectively. Similarly 2,6-dichloropurine was coupled with bromo sugar 9 to obtain the corresponding nucleoside as an anomeric mixture (2:1, β:α ratio) in 64% yield. Both anomers were separated by preparative TLC to provide 18 and 18α as white foams. Separate treatment of 18 and 18α with sodium azide in aqueous ethanol at reflux produced the corresponding 2,6-diazido intermediates 19 and 19α, which were subjected to reduction with Pd/C to afford blocked diaminopurine nucleosides 20 and 20α, respectively. Deblocking of 20 and 20α with NaOMe produced the target 2-aminoadenine nucleosides 21 and 21α. Conversion of 21 to the guanine nucleoside 22 was accomplished by treatment with adenosine deaminase.^{[21]} Though the deamination was slow, it went to completion at room temperature in 68 hours. The 2-chloroadenine nucleosides 24 (84%) and 24α (75%) were prepared by first converting the dichloropurine nucleosides 18 and 18α to their 6-methoxy intermediate 23 with sodium methoxide followed by treatment with ethanolic ammonia.^{[8]}

### Biological Results

### In Vitro Cytotoxicity

The concentration of compound required to inhibit cell growth by 50% (IC₅₀) after 72 hours of incubation was determined for each unprotected analog with eight human tumor cell lines (SNB-7 CNS, DLD-1 colon, CCRF-CEM leukemia, NCI-H23 NSCL, ZR-75-1 breast, LOX melanoma, PC-3 prostate, and CAKI-1 renal). The most active compound in this series was methyl-F-araC (13, Table 1), which was found to have significant cytotoxicity against four of the cell lines in the panel. The purine analogs demonstrated modest cytotoxicity against the solid tumor cell lines (IC₅₀'s between 5 and 80 μM), while the uracil and thymine analogs were not active against any cell line (IC₅₀'s greater than 200 μM). The α-anomers of these compounds were also screened but were not found to be cytotoxic (data not shown).

CCRF-CEM cells are a T-cell leukemia cell line that is known to be very sensitive to nucleoside analogs. Methyl-F-araC was a very potent inhibitor of this cell line with an IC₅₀ of 0.012±0.003 μM. CCRF-CEM cell growth was also inhibited by the 2-Cl-adenine (24), 2,6-diaminopurine (21), and guanine (22) analogs with IC₅₀'s of approximately 0.5 μM. The inhibition of CCRF-CEM cell growth caused by either methyl-F-araC or the 2-Cl-adenine analog (4′-C-methyl-clofarabine, 24) was prevented by adding dCyd to the culture medium and neither compound was active in cells that lacked dCyd kinase. These results indicated that dCyd kinase was the primary enzyme responsible for the initial activation step of these two agents in CCRF-CEM cells. The cytotoxicity of the diaminopurine analog 21 was prevented by the addition of deoxycoformycin, a potent inhibitor of adenosine deaminase, which indicated that 21 was deaminated to the dGuo analog before conversion to cytotoxic nucleotides.

### In Vitro Metabolic Studies in CCRF-CEM Cells

CCRF-CEM cells were incubated with methyl-F-araC, araC, and gemcitabine, and the amount of intracellular triphosphate (TP) of each compound was determined. There was significant metabolism of each of these compounds, and their triphosphates did not co-elute with any of the natural nucleotides (ATP, GTP, CTP, or UTP). Incubation of CCRF-CEM cells with 100 nM of each compound for two hours resulted in an intracellular concentration of methyl-F-araC-TP (16±2 pmoles/10⁶ cells) that was similar to those of araC-TP (12±1 pmoles/10⁶ cells) and gemcitabine-TP (17±3 pmoles/10⁶ cells) (mean±SD, N=3). These results indicated that methyl-F-araC was a good substrate for deoxycytidine kinase. The intracellular half-life of each triphosphate was similar: methyl-F-araC-TP (7.1 hours, N=2); araC-TP (5.6 hours, N=2); gemcitabine-TP (5.0 hours, N=2).

### In Vivo Activity

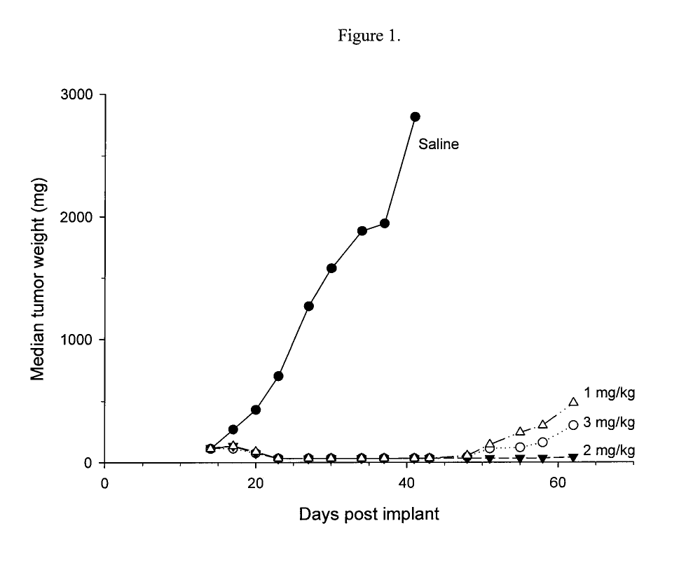

Because of its potent in vitro activity, methyl-F-araC (13) was evaluated for in vivo activity against three solid tumor xenografts (CAKI-1 renal, NCI-H23 NSCL, and LOX melanoma). Prior to these studies the maximally tolerated dose of methyl-F-araC was determined to be 3 mg/kg given once per day for 9 consecutive days. Methyl-F-araC demonstrated excellent activity against the CAKI-1 tumors (FIG. 1). Female NCr-nu athymic mice were implanted subcutaneously with CAKI-1 tumor fragments. When tumors were approximately 100-250 mg, mice were treated ip with methyl-F-araC at 1, 2, or 3 mg/kg/dose (1 treatment each day for 9 consecutive days starting on day 14). Each treatment group contained 6 mice. The tumors were measured with calipers twice each week, and the weight (mg) was calculated. In this experiment there were 3 of 6 tumor-free survivors at the end of the experiment (62 days post implant) in each treatment group. Good results were also seen against NCI-H23 and LOX human tumor xenografts (Table 2). Therefore, methyl-F-araC demonstrated good to excellent in vivo antitumor activity in the three solid tumor xenografts that have been tested to date.

TABLE 1
Cytotoxicity data of methyl-F-araC

| Cell line | IC₅₀ (μM) |
|---|---|
| SNB-7 | >200 |
| DLD-1 | >200 |
| CCRF-CEM | 0.012 ± 0.003 |
| NCI-H23 | 0.19 ± 0.01 |
| ZR-75-1 | >200 |
| LOX | 0.24 ± 0.15 |
| PC-3 | >200 |
| CAKI-1 | 0.54 ± 0.58 |

TABLE 2
Response of subcutaneously implanted human
tumor xenografts to methyl-F-araC (13)

| | Optimal | | | |
|---|---|---|---|---|
| | i.p. dosage | Tumor size | T-C | Tumor-free |
| Tumor | (mg/kg/dose) | range (mm) | (days) | survivors |
| CAKI-1 renal | 3 | 100-245 | >34.6^{a} | 3/6 |
| NCI-H23 NSCL | 4 | 100-270 | 18.4^{b} | 0/5 |
| LOX melanoma | 3 | 100-221 | 15.0^{c} | 0/6 |

Xenografts were implanted sc on the flanks of female nude mice. When tumors were approximately 100-250 mg, they were treated ip with 3 or 4 mg/kg/dose of methyl-F-araC (q1d×9) and tumor size was measured twice weekly thereafter. Tumor-free survivors are the number of mice that were tumor-free at the end of the experiment/total number of mice in the treatment group.
^{a}The difference in the median of times poststaging for tumors of the treated (T) and control (C) groups to double in mass three times.
^{b}The difference in the median of times poststaging for tumors of the treated (T) and control (C) groups to double in mass two times.
^{c}The difference in the median of times poststaging for tumors of the treated (T) and control (C) groups to double in mass four times.

1-(4-C-Methyl-2-fluoro-β-D-arabinofuranosyl) cytosine (13) was found to be highly cytotoxic and had significant antitumor activity in mice implanted with human tumor xenografts. This compound is a substrate for deoxycytidine kinase and significant levels of its 5′-triphosphate accumulated in CCRF-CEM cells.

### EXPERIMENTAL

TLC analysis was performed on Analtech precoated (250 μm) silica gel GF plates. Melting points were determined on a Mel-Temp apparatus and are uncorrected. Purifications by flash chromatography were carried out on Merck silica gel (230-400 mesh). Evaporations were performed with a rotary evaporator, higher boiling solvents (DMF, pyridine) were removed in vacuo (<1 mm, bath to 35° C.). Products were dried in vacuo (<1 mm) at 22-25° C. over P₂O₅. The mass spectral data were obtained with a Varian-MAT 311A mass spectrometer in the fast atom bombardment (FAB) mode or with a Bruker BIOTOF II by electrospray ionization (ESI). ¹HNMR spectra were recorded on a Nicolet NT-300 NB spectrometer operating at 300.635 MHz. Chemical shifts in CDCl₃ and Me₂SO-d₆ are expressed in parts per million downfield from tetramethylsilane (TMS), and in D₂O chemical shifts are expressed in parts per million downfield from sodium 3-(trimethylsilyl)propionate-2,2,3,3-d₄ (TMSP). Chemical shifts (δ) listed for multiplets were measured from the approximate centers, and relative integrals of peak areas agreed with those expected for the assigned structures. UV absorption spectra were determined on a Perkin-Elmer lambda 9 spectrophotometer by dissolving each compound in MeOH or EtOH and diluting 10-fold with 0.1 N HCl, pH 7 buffer, or 0.1 N NaOH. Numbers in parentheses are extinction coefficients (ε×10⁻³). Microanalyses were performed by Atlantic Microlab, Inc. (Atlanta, Ga.) or the Spectroscopic and Analytical Department of Southern Research Institute. Analytical results indicated by element symbols were within ±0.4% of the theoretical values, and where solvents are indicated in the formula, their presence was confirmed by ¹HNMR.

### Cell Culture Cytotoxicity

All cell lines were grown in RPMI 1640 medium containing 10% fetal bovine serum, sodium bicarbonate, and 2 mM L-glutamine. For in vitro evaluation of the sensitivity of these cell lines to compounds, cells were plated in 96-well microtiter plates and then were exposed continuously to various concentrations of the compounds for 72 h at 37° C. Cell viability was measured using the MTS assay [3-(4,5-dimethylthiazol-2-yl)-5-(3-carboxymethoxyphenyl)-2-(4-sulfophenyl)-2H-tetrazolium, inner salt; MTS and an electron coupling reagent (phenazine ethosulfate; PES)]. Absorbance was read at 490 nm. The background absorbance mean was subtracted from the data followed by conversion to percent of control. The drug concentrations producing survival just above and below 50% level were used in a linear regression analysis to calculate the IC₅₀.

### Measurements of Intracellular Triphosphates

CCRF-CEM cell extracts were collected by centrifugation and resuspended in ice-cold 0.5 M perchloric acid. The samples were centrifuged at 12,000×g, and the supernatant fluid was neutralized and buffered by adding 4 M KOH and 1 M potassium phosphate, pH 7.4. KClO₄ was removed by centrifugation, and a portion of the supernatant fluid was injected onto a strong anion exchange HPLC (Bio Basic anion exchange column, Thermo Electron Corp., Bellefonte, Pa.). Nucleotides were eluted with a 30-min linear salt and pH gradient from 6 mM ammonium phosphate (pH 2.8) to 900 mM ammonium phosphate (pH 6). Peaks were detected as they eluted from the column by their absorbance at 254 nm.

### Experimental Chemotherapy

Mice, obtained from various commercial suppliers, were housed in microisolator cages and were allowed commercial mouse food and water ad libitum. The three human tumors were obtained from the Developmental Therapeutics Program Tumor Repository (Frederick, Md.) and were maintained in in vivo passage. Only tumor lines that tested negative for selected viruses were used. For the in vivo evaluation of the sensitivity of human tumors to the compounds, female NCr-nu athymic mice were implanted subcutaneously (sc) with 30-40 mg tumor fragments. In each experiment, methyl-F-araC (13) was tested at three dosage levels. Procedures were approved by the Southern Research Institutional Animal Care and Use Committee, which conforms to the current Public Health Service Policy on Humane Care and Use of Laboratory Animals and the Guide for the Care and Use of Laboratory Animals.

Antitumor activity was assessed on the basis of delay in tumor growth (T-C). The delay in tumor growth is the difference in the median of times post staging for tumors of the treated and control groups to double in mass two, three, or four times. Drug deaths and any other animal whose tumor failed to attain the evaluation size were excluded. Tumors were measured in two dimensions (length and width) twice weekly, and the tumor weight was calculated using the formula (length×width²)/2 and assuming unit density. The mice were also weighed twice weekly.

The following non-limiting examples are presented to further illustrate the present invention.

### Example 1

Methyl 4-C-(p-Anisyldiphenylmethoxymethyl)-2-deoxy-2-fluoro-β-D-arabinofuranoside (2a) and Methyl 5-(p-Anisyldiphenylmethoxymethyl)-4-C-hydroxymethyl-2-deoxy-2-fluoro-β-D-arabinofuranoside (2b). To a solution of 1 (342 mg, 1.75 mmol) in dry pyridine (15 mL) was added in one portion solid 97% p-anisylchlorodiphenylmethane (816 mg, 2.56 mmol). The reaction mixture was stirred at room temperature for 20 hours and then evaporated. The resulting residue was co-evaporated with two portions of toluene before being purified by flash chromatography on silica gel (45 g) using a gradient from CHCl₃ to 97:3 CHCl₃/MeOH. The first eluted fraction gave 2a ((246 mg, 30%) as a white foam: TLC 97:3 CHCl₃/MeOH, R_{f} 0.45; MS m/z 491 (m+Na)⁺; ¹H NMR (CDCl₃) 7.20-7.45 (m, 12H, aromatic H's), 6.86-6.88 (m, 2H, para-H's of 4-methoxyphenyl), 5.10-5.33 (m, 2H, H-1 and H-2), 4.44-4.52 (m, 1H, H-3), 3.81 (s, 3H, OCH₃ of p-methoxyphenyl), 3.56 (s, 3H, 1-OCH₃), 3.46-3.58 (m, 3H, two 4-C-hydroxymethyl and one 5-CH₂ hydrogens), 3.04 (d, 1H, 5-CH₂, J=12 Hz), 2.95 (d, 1H, 3-OH, J=12 Hz), 2.18 (t, 1H, 5-OH, J=8 Hz). The second fraction provided 2b ((137 mg, 17%): TLC 97:3 CHCl₃/MeOH, R_{f} 0.39; MS m/z 491 (m+Na)⁺; ¹H NMR (CDCl₃) 7.20-7.48 (m, 12H, aromatic H's), 6.82-6.86 (m, 2H, para-H's of 4-methoxyphenyl), 4.83-5.06 (m, 1H, H-2), 4.92 (dd, 1H, H-1, J=2 and 6 Hz), 4.3-4.40 (m, 1H, H-3), 3.94-4.02 (m. 1H-5-CH₂), 3.82-4.02 (m, 1H, 5-CH₂), 3.80 (s, 3H, OCH₃ of p-methoxyphenyl), 3.25 (s, 3H, 1-OCH₃), 3.26-3.28 (m, 1H, 4-C-hydroxymethyl), 3.18-3.20 (m, 1H, 4-C-hydroxymethyl), 2.80 (d, 1H, 3-OH, J=12 Hz), 2.12 (t, 1H, 5-OH, J=8 Hz).

### Example 2

Methyl 4-C-(p-Anisyldiphenylmethoxymethyl)-3,5-di-O-benzoyl-2-deoxy-2-fluoro-β-D-arabinofuranoside (3). To a solution of 2a (52 mg, 0.11 mmol) in dry pyridine (5 mL) at 0° C. was added benzoyl chloride (91 μl, 0.77 mmol) dropwise. After 5 minutes, the cooling bath was removed, and stirring was continued for 18 hours. The solution was evaporated to a solid that was co-evaporated once with toluene. The solid was purified by silica gel preparative TLC (Analtech GF, 10×20 cm, 1000μ) with 3:1 hexane/EtOAc as solvent to give 3 (69 mg, 92%) as a white foam: TLC 3:1 hexane/EtOAc, R_{f} 0.44; MS m/z 699 (m+Na)⁺; ¹H NMR (CDCl₃) 7.82-7.92 (m, 4H, ortho H's of benzoyl), 7.50-7.62 (m, 2H, para-H's of benzoyl), 7.12-7.92 (m, 16H, aromatic H's), 6.63-6.68 (m, 2H, p-H's of 4-methoxyphenyl), 6.02 (dd, 1H, H-3, J=8 and 10 Hz), 5.46-5.70 (m, 1H, H-2), 5.18 (bd, 1H, H-1, J=6 Hz), 4.50-4.60 (m, 2H, 5CH₂), 3.70 (s, OCH₃ of p-methoxyphenyl), 3.48 (s, 3H, 1-OCH₃), 3.36-3.42 (m, 1H, 4-CH₂), 3.14-3.18 (m, 1H, 4-CH₂).

### Example 3

Methyl 3,5-Di-O-benzoyl-2-deoxy-2-fluoro-4-C-hydroxymethyl-β-D-arabinofuranoside (4). A solution of 3 (576 mg, 0.85 mmol) in 4:1 acetic acid/water (20 mL) was stirred at room temperature for 19 hours and then evaporated. The residue obtained was partitioned between EtOAc and ice-cold saturated NaHCO₃. The aqueous layer was extracted twice with EtOAc, and the combined organic layers were washed with saturated NaCl, dried (MgSO₄), and evaporated. The residue was purified by flash chromatography on silica gel (20 g) using a gradient from hexane to 2:1 hexane/EtOAc to give 4 (306 mg, 89%) as a clear syrup: TLC 2:1 hexane/EtOAc, R_{f} 0.29; MS m/z 405 (m+H)⁺; ¹H NMR (CDCl₃) 8.0-8.1 (m, 4H, ortho H's of benzoyl), 7.34-7.64 (m, 6H, para and meta H's of benzoyl), 6.08 (dd, 1H, H-3, J=8 and 18 Hz), 5.27-5.50 (m, 1H, H-2), 5.12 (dd, 1H, H-1, J=2 and 8 Hz), 4.52-4.65 (m, 2H, 5-CH₂), 3.76 (s, 2H, 4-CH₂), 3.50 (s, 3H, OCH₃), 2.12 (bh, 1H. 5-OH).

### Example 4

Methyl 3,5-Di-O-benzoyl-2-deoxy-2-fluoro-4-C-phenoxythiocarbonyloxymethyl-β-D-arabinofuranoside (5). To a solution of 4 (75 mg, 0.19 mmol) and 4-(dimethylamino)pyridine (93 mg, 0.75 mmol) in dry MeCN (7 mL) at room temperature was added dropwise phenyl chlorothionoformate (39 μl, 0.28 mmol). The resulting yellow solution was stirred at room temperature for 3 hours and then evaporated. The residue obtained was partitioned between ice-cold 5% citric acid and EtOAc. The aqueous layer was extracted twice with EtOAc, and the combined organic layers were washed with water, dried (MgSO₄), and evaporated to a gum. This crude 5 was purified by silica gel preparative TLC (Analtech GF, 10×20 cm, 1000μ) with 3:1 hexane/EtOAc as solvent to obtain pure 5 (92 mg, 90%) as a white foam: TLC 3:1 hexane/EtOAc, R_{f} 0.55; MS m/z 563 (m+Na)⁺; ¹H NMR (CDCl₃) 8.0-8.10 (m, 4H, ortho H's of benzoyl), 7.26-7.68 (m, 9H, aromatic H's), 6.90-6.94 (m, 2H, ortho H's of phenyl), 6.10 (dd, 1H, H-1, J=8 and 18 Hz), 5.16-5.42 (m, 1H, H-2), 5.10 (dd, 1H, H-3, J=2 and 8 Hz), 4.62-4.74 (m, 4H, 4 and 5-CH₂), 3.52 (s, 3H, OCH₃).

### Example 5

Methyl 3,5-Di-O-benzoyl-2-deoxy-2-fluoro-4-C-methyl-β-D-arabinofuranoside (6). A solution of 5 (4.0 g, 7.4 mmol) in anhydrous toluene (125 mL) was purged with argon before solid 98% 1,1′-azobis(cyclohexanecarbonitrile) (657 mg, 2.6 mmol) was added in one portion. The argon purge was repeated followed by a syringe addition of 97% tris(trimethylsilyl) silane (10 mL, 31 mmol) over 5 minutes. The reaction solution was warmed over 0.5 hours to 100° C., maintained at 100° C. for 5 hours, cooled to room temperature. and reduced under vacuum to an oil. The crude product was purified by column chromatography on silica gel with 5:1 cyclohexane/EtOAc as solvent to provide 6 (2.4 g, 84%) as a clear oil: TLC 85:15 cyclohexane/EtOAc, R_{f} 0.38; MS m/z 389 (m+H)⁺; ¹H NMR (CDCl₃) 8.08-8.12 (m, 4H, ortho H's of benzoyl), 7.38-7.66 (m, 6H, para and meta H's of benzoyl), 6.28 (dd, 1H, H-3, J=8 and 18 Hz), 5.15-5.37 (m, 1H, H-2), 5.04 (dd, 1H, H-1, J=2 and 8 Hz), 4.46-4.62 (m, 2H, 5-CH₂), 3.44 (s, 3H, OCH₃), 1.34 (s, 3H, CH₃).

### Example 6

3,5-Di-O-benzoyl-2-deoxy-2-fluoro-4-C-methyl-α,β-D-arabinofuranoside (7). A solution of 6 (1.3 g, 3.35 mmol) in 9:1 trifluoroacetic acid/water (23 mL) was maintained at 60-65° C. for 24 hours, cooled to room temperature, and diluted with CH₂Cl₂ (100 mL). The solution was added dropwise to a stirred mixture of ice (300 g) and saturated NaHCO₃ (300 mL). Solid NaHCO₃ was added during the addition to maintain a pH of 7. The mixture was extracted with CH₂Cl₂ (3×100 mL), and the organic extract was washed with water (2×50 mL), dried (MgSO₄), and concentrated to a syrup (1.3 g). This material was flash chromatographed on silica gel (100 g) with 3:1 hexane/EtOAc as solvent to yield pure 7 (1.05 g, 83%) as a white solid: TLC 3:1 hexane/EtOAc, R_{f} 0.40; MS m/z 375 (m+H)⁺; ¹H NMR (CDCl₃) 8.04-8.12 (m, 4H, ortho H's of benzoyl), 7.40-7.64 (m, 6H, para and meta H's of benzoyl), 5.70-5.88 (m, 1H, H-3α,β), 5.65 (dd, 0.65H, H-1α, J=12 and 4 Hz), 5.52-5.57 (m, 0.35H, H-1β), 5.07-5.28 (m, 1H, H-2α,β), 4.34-4.78 (m, 2H, 5-CH₂), 3.52 (d, 0.35H, 1-OH β), 2.79 (dd, 0.65H, 1-OH α, J=1 and 4 Hz), 1.51 (s, 2H, CH₃α), 1.36 (s, 1H, CH₃β).

### Example 7

1-O-Acetyl-3,5-di-O-benzoyl-2-deoxy-2-fluoro-4-C-methyl-α,β-D-arabinofuranose (8). To a solution of 7 (1.04 g, 2.78 mmol) in dry pyridine (25 mL) at 5° C. was added dropwise acetic anhydride (0.79 mL, 8.37 mmol) over 5 minutes. After 15 minutes, the solution was allowed to warm to room temperature where it was held for 18 hours. The reaction solution was concentrated in vacuum and co-evaporated with toluene (3×2 mL). The crude product was purified by flash chromatography on silica gel (70 g) using 3:1 hexane/EtOAc as solvent to provide pure 8 (1.06 g, 91%) as a white solid: TLC 3:1 hexane/EtOAc, R_{f} 0.50; MS m/z 439 (m+Na)⁺; ¹H NMR (CDCl₃) 8.06-8.12 (m, 4H, ortho H's of benzoyl), 7.40-7.68 (m, 6H, para and meta H's of benzoyl), 6.41-6.46 (m, 1H, H-1α,β), 5.16 (m, 1H, H-3α,β), 5.16-5.48 (m, 1H, H-2α,β), 4.40-4.64 (m, 2H, 5-CH₂), 2.16 (s, 2.25H, CH₃ of 1-O-acetyl, α), 2.0 (s, 0.75H, CH₃ of 1-O-acetyl, β), 1.50 (s, 2.25H, CH₃α), 1.40 (s, 0.75H, CH₃β).

### Example 8

3,5-Di-O-benzoyl-2-deoxy-2-fluoro-4-C-methyl-α,β-D-arabinofuranosyl Bromide (9). A solution of 8 (507 mg, 1.22 mmol) in CH₂Cl₂ (35 mL) was stirred with MgSO₄ for 1.5 hours, filtered, and evaporated to a stiff syrup. After being dried under vacuum for 2 hours, the residue was dissolved in anhydrous CH₂Cl₂ (20 mL), chilled to 5° C., and treated dropwise with 33% HBr in acetic acid (5.5 mL) The clear yellow solution in a tightly sealed flask was placed in a nitrogen filled bag, refrigerated for 20 hours, and evaporated in vacuum. The dark orange highly reactive residue was co-evaporated with toluene (2×3 mL) and then used directly in the various pyrimidine and purine couplings: TLC 3:1 hexane/EtOAc, R_{f} 0.85.

### Example 9

N⁴-Benzoyl-1-(3,5-di-O-benzoyl-2-deoxy-2-fluoro-4-C-methyl-β-D-arabinofuranosyl)cytosine (10). A suspension of 98% N⁴-benzoyl cytosine (863 mg, 3.93 mmol) in dry MeCN (20 mL) at room temperature was treated dropwise with 95% N,O-bis (trimethylsilyl)acetamide (BSA) (3.6 mL) and stirred for 2 hours. The clear solution obtained was evaporated in vacuum to a fluid oil that was dried under vacuum for an additional 2 hours before being dissolved in ClCH₂CH₂Cl (20 mL). To this solution was added in one portion a solution of 9 [prepared from 8 (507 mg, 1.22 mmol)] in ClCH₂CH₂Cl (10 mL). The reaction solution was heated at 100° C. for 4 hours, cooled, and quenched with MeOH (15 mL) at 5° C. The reaction was stirred at room temperature for ½ hour before being filtered through a Celite pad to remove excess pyrimidine. The solid was washed with CHCl₃ and MeCN until free of 10, and the combined filtrate and washings were evaporated to a yellow solid. This crude product was purified by flash chromatography on silica gel (45 g) with 1:1 hexane/EtOAc as solvent to give pure 10 (336 mg, 48%) as a white solid: TLC 1:1 hexane/EtOAc, R_{f} 0.28; MS m/z 572 (m+H)⁺; ¹H NMR (CDCl₃) 8.70 (bh, 1H, NH), 7.86-8.14 (m, 7H, H-6 and ortho H's of benzoyl), 7.46-7.70 (m, 10H, H-5 and para and meta H's of benzoyl), 6.47 (dd, 1H, H-1′, J=4 and 20 Hz), 5.88 (dd, 1H, H-3′, J=0.5 and 18 Hz), 5.52 (dd, 1H, H-2′, J=4 and 50 Hz), 4.58-4.68 (m, 2H, 5′-CH₂), 1.50 (s, 3H, 4′-CH₃).

From impure fractions, the α-anomer 10α (41 mg, 6%) was recovered as a white solid by silica gel preparative TLC (Analtech GF, 10×20 cm, 500μ) with 99:1 CHCl₃/MeOH as solvent: TLC 1:1 hexane/EtOAc, R_{f} 0.45; MS m/z 572 (m+H)⁺; ¹H NMR (CDCl₃) 8.80 (bh, 1H, NH), 7.40-8.16 (m, 17H, H-5, H-6 and aromatic H's), 6.28 (dd, 1H, H-1′, J=1 and 18 Hz), 5.90 (dd, 1H, H-3′, J=1 and 14 Hz), 5.46 (dd, 1H, H-2′, J=1 and 48 Hz), 4.44-4.50 (m, 2H, 5′-CH₂), 1.64 (s, 3H, 4′-CH₃).

### Example 10

1-(3,5-Di-O-benzoyl-2-deoxy-2-fluoro-4-C-methyl-β-D-arabinofuranosyl)uracil (11). A suspension of uracil (55 mg, 0.49 mmol) in dry MeCN (3 mL) at room temperature was treated dropwise with 95% N,O-bis(trimethylsilyl)acetamide (BSA) (0.44 mL) and stirred for 1 hour. The clear solution obtained was evaporated under vacuum to a syrup that was dried for an additional 1 hour before being dissolved in ClCH₂CH₂Cl (3 mL). To this solution was added in one portion a solution of 9 [prepared from 8 (57 mg, 0.14 mmol)] in ClCH₂CH₂Cl (2 mL), and the mixture was heated at 100° C. for 4 hours, cooled, and quenched with MeOH (1 mL) at 5° C. After being stirred at room temperature for 1.5 hours, the mixture was filtered through a Celite pad to remove excess uracil, and the filtrate was concentrated to a yellow solid. This anomeric mixture was resolved by preparative TLC (Analtech GF, 10×20 cm, 1000μ) with multiple development in 97:3 CHCl₃/MeOH to provide 11 (42 mg, 65%) and 11α (4 mg, 6%) as white solids. 11: TLC 97:3 CHCl₃/MeOH, R_{f} 0.50; MS m/z 469 (m+H)⁺; ¹H NMR (CDCl₃). 8.32 (bs, 1H, H-3), 8.06-8.14 (m, 4H, ortho H's of benzoyl), 7.44-7.70 (m, 7H, H-6 and para and meta H's of benzoyl), 6.37 (dd, 1H, H-1′, J=4 and 20 Hz), 5.86 (dd, 1H, H-5, J=2 and 18 Hz), 5.66 (bd, 1H, H-3′, J=8 Hz), 5.32 (ddd, 1H, H-2′, J=2, 4 and 50 Hz), 4.56-4.64 (m, 2H, 5′-CH₂), 1.46 (s, 3H, 4′-CH₃). 11α: TLC 97:3 CHCl₃/MeOH, R_{f} 0.46; MS m/z 469 (m+H)⁺; ¹H NMR (CDCl₃). 8.32 (bs, 1H, H-3), 7.98-8.14 (m, 4H, ortho H's of benzoyl), 7.44-7.68 (m, 7H, H-6 and para and meta H's of benzoyl), 6.24 (dd, 1H, H-1′, J=3 and 16 Hz), 5.80-5.92 (m, 2H, H-3′ and H-5), 5.41 (dt, 1H, H-2′, J=2 and 50 Hz), 4.64-4.68 (m, 1H, 5′-CH₂), 4.40-4.46 (m, 1H, 5′-CH₂), 1.52 (s, 3H, 4′-CH₃).

### Example 11

1-(3,5-Di-O-benzoyl-2-deoxy-2-fluoro-4-C-methyl-β-D-arabinofuranosyl)thymine (12). Compound 9 [prepared from 8 (60 mg, 0.14 mmol)] was treated with 99% thymine (62 mg, 0.48 mmol) as described for 11 to give 12 (43 mg, 62%) and 12α (4 mg, 6%) as white solids. 12: TLC 98:2 CHCl₃/MeOH, R_{f} 0.43; MS m/z 483 (m+H)⁺; ¹H NMR (CDCl₃) 8.24 (bs, 1H, H-3), 7.44-7.70 (m, 10H, aromatic H's), 7.34 (s, 1H, H-6), 6.38 (dd, 1H, H-1′, J=4 and 20 Hz), 5.88 (dd, 1H, H-3′, J=2 and 18 Hz), 5.31 (ddd, 1H, H-2′, J=2, 4 and 50 Hz), 4.62 (s, 2H, 5′-CH₂), 1.74 (s, 3H, 5-CH₃), 1.46 (s, 3H, 4′-CH₃). 12α: TLC 98:2 CHCl₃/MeOH, R_{f} 0.39; MS m/z 483 (m+H)⁺; ¹H NMR (CDCl₃) 8.36 (bs, 1H, H-3), 7.46-7.68 (m, 10H, aromatic H's), 7.30 (s, 1H, H-6), 6.28 (dd, 1H, H-1′, J=3 and 16 Hz), 5.87 (dd, 1H, H-3′, J=3 and 16 Hz), 5.40 (dt, 1H, H-2′, J=2 and 50 Hz), 4.40-4.66 (m, 2H, 5′-CH₂), 1.98 (s, 3H, 5-CH₃), 1.52 (s, 3H, 4′-CH₃).

### Example 12

1-(2-Deoxy-2-fluoro-4-C-methyl-β-D-arabinofuranosyl)cytosine (13).^{[2]} A suspension of 10 (334 mg, 0.58 mmol) in MeOH (30 mL) at room temperature. was treated dropwise with 0.5M sodium methoxide in MeOH (0.58 mL). The solid dissolved after 15 minutes, and the solution was stirred for 2 hours, neutralized to pH 7 with glacial acetic acid, and evaporated to an oil. Preparative TLC on silica gel (Analtech GF, 20×20 cm, 2000μ) with 3:1:0.1 CHCl₃/MeOH/concentrated NH₄OH as eluant provided 13 as a hygroscopic white foam. This residue was dissolved in 2-PrOH (10 mL), 1.0 M HCL in ether (1.17 mL) was added and the mixture was evaporated. From a suspension of this material in acetone was recovered hydrochloride 13 (157 mg, 89%) as a white solid: m.p. 230-231° C.; TLC 3:1:0.1 CHCl₃/MeOH/NH₄OH, R_{f} 0.40; HPLC 99%, t_{R}=8.6 min, 9:1 NH₄H₂PO₄ (0.01M, pH 5.1)/MeOH; MS m/z 260 (M+H)⁺; UV λmax pH 1, 279 (13.8), pH 7, 269 (9.4), pH 13, 271 (9.6); ¹H NMR (DMSO-d₆) 9.70 (s, 1H, 4-NH₂), 8.62 (s, 1H, 4-NH₂), 8.24 (d, 1H, H-6, J=8 Hz), 6.10-6.17 (m, 2H, H-1′ and H-5 overlapped), 5.95 (bh, 1H₂OH), 5.23 (dt, 1H, H-2′, J=4 and 52 Hz), 4.26 (dd, 1H, H-3′, J=4 and 20 Hz), 3.40-3.50 (m, 3H, 5′-CH₂ and OH), 1.25 (s, 3H, 4′-CH₃). Anal. Calcd. For C₁₀H₁₄FN₃O₄.HCL0.10C₃H₈O, 0.20H₂O: C, 40.52; H, 5.35; N, 13.76. Found: C, 40.82; H, 5.18; N, 13.58.

The α-anomer was prepared from 10α as described for 13 except the HCl salt formation was omitted. Pure 13α (77%) was obtained from acetone as a white solid: m.p. 222-223° C.; TLC 3:1:0.1 CHCl₃/MeOH/NH₄OH, R_{f} 0.40; HPLC 100%, t_{R}=7.7 min, 9:1 NH₄H₂PO₄ (0.01M, pH 5.1)/MeOH; MS m/z 260 (M+H)⁺; UV λmax pH 1, 278 (13.3), pH 7, 270 (9.3), pH 13, 271 (9.3); ¹H NMR (DMSO-d₆) 7.59 (d, 1H, H-6), 7.28 (bs, 1H, 4-NH₂), 7.20 (bs, 1H, 4-NH₂), 6.0 (dd, 1H, H-1′ J=4 and 18 Hz), 5.74-5.78 (m, 2H, H-5 and 3′-OH overlapped), 4.9-5.1 (m, 2H, H-2′ and 5′-OH overlapped), 4.24 (dt, 1H, H-3′, J=3 and 18 Hz), 3.30-3.40 (m, 2H, 5′-CH₂), 1.24 (s, 3H, 4′-CH₃). Anal. Calcd. For C₁₀H₁₄FN₃O₄: C, 46.33; H, 5.44; N, 16.21. Found: C, 46.19; H, 5.23; N, 16.09.

### Example 13

1-(2-Deoxy-2-fluoro-4-C-methyl-β-D-arabinofuranosyl)uracil (14).^{[2]} Compound 14 was prepared from 11 using the method described for 13α. Pure 14 (77%) was obtained as a clear glass from acetone and was subsequently ground to a white powder: m.p. 70-75° C.; TLC 3:1:0.1 CHCl₃/MeOH/NH₄OH, R_{f} 0.62; HPLC 99%, t_{R}=6.4 min, 85:15 NH₄H₂PO₄ (0.01M, pH 5.1)/MeOH; MS m/z 261 (M+H)⁺; UV λmax pH 1, 261 (10.5), pH 7, 261 (10.3), pH 13, 261 (8.0); ¹H NMR (DMSO-d₆) 11.42 (bh, 1H, H-3), 7.86 (d, 1H, H-6, J=8 Hz), 6.20 (dd, 1H, H-1′, J=4 and 12 Hz), 5.92 (bs, 1H, 3′-OH), 5.72 (d, 1H, H-5, J=8 Hz), 5.23 (dt, 2H, H-2′, J=4 and 52 Hz and 5′-OH overlapped and exchanged with D2O), 4.28 (dd, 1H, H-3′, J=4 and 22 Hz), 3.40-3.44 (m, 2H, 5′-CH₂), 1.10 (s, 3H, 4′-CH₃). Anal. Calcd. For C₁₀H₁₃FN₂O₅:0.25H₂O: C, 45.37; H, 5.14; N, 10.58. Found: C, 45.32; H, 4.89; N, 10.42.

### Example 14

1-(2-Deoxy-2-fluoro-4-C-methyl-β-D-arabinofuranosyl)thymine (15). Compound 15 was synthesized from 12 using the conditions described for 13α but with a 7 hour reaction time and with 5:1 CHCl₃/MeOH+1% concentrated NH₄OH as eluent for preparative TLC. Pure 15 (92%) was recovered from acetone (as for 14) as a white powder: m.p. 80° C.; TLC 5:1 CHCl₃/MeOH+1% NH₄OH, R_{f} 0.52; HPLC 100%, t_{R}=5.2 min, 3:1 NH₄H₂PO₄ (0.01M, pH 5.1)/MeOH; MS m/z 275 (M+H)⁺; UV λmax pH 1, 267 (9.9), pH 7, 267 (9.8), pH 13, 266 (7.8); ¹H NMR (DMSO-d₆) 11.40 (s, 1H, H-3), 7.74 (s, 1H, H-6), 6.14 (dd, 1H, H-1′, J=6 and 12 Hz), 5.88 (bd, 1H, 3′-OH, J=6 Hz), 5.3-5.4 (m, 1H, 5′-OH), 5.18 (dt, 1H, H-2′, J=4 and 52 Hz), 4.31 (dt, 1H, H-3′, J=4 and 22 Hz), 3.40-3.48 (m, 2H, 5′-CH₂), 1.80 (s, 3H, 5-CH₃), 1.08 (s, 3H, 4′-CH₃). Anal. Calcd. For C₁₁H₁₅FN₂O5.0.50H₂O: C, 46.64; H, 5.69; N, 9.89. Found: C, 46.54; H, 5.33; N, 9.68.

### Example 15

6-Chloro-9-(3,5-di-O-benzoyl-2-deoxy-2-fluoro-4-C-methyl-β-D-arabinofuranosyl)purine (16). A suspension of 98% 6-chloropurine (102 mg, 0.65 mmol) in dry MeCN (5 mL) at room temperature was treated in one portion with 60% NaH (35 mg, 0.88 mmol). The mixture was stirred 40 minutes before the immediate addition of 9 [prepared from 8 (177 mg, 0.43 mmol)] dissolved in MeCN (2 mL). After 6 hours, the stirred mixture was adjusted to about pH 6 with glacial acetic acid, and stirring was continued 15 minutes before the solids present were collected, washed with MeCN, and discarded. The combined filtrate and washings were evaporated to a yellow residue that was purified by silica gel preparative TLC (Analtech GF, 20×20 cm, 2000μ) with 99:1 CHCl₃/MeOH as eluent. The recovered anomeric product was resolved by preparative TLC developed twice in 2:1 hexane/EtOAc to provide 16 (79 mg, 36%) and 16α (31 mg, 14%) as white solids: 16, TLC 2:1 hexane/EtOAc, R_{f} 0.40; MS m/z 511 (M+H)⁺; ¹HNMR (CDCl₃) 8.78 (s, 1H, H-2), 8.42 (d, 1H, H-8, J=4 Hz), 8.10-8.16 (m, 4H, ortho H's of benzoyl), 7.46-7.72 (m, 6H, para and meta H's of benzoyl), 6.76 (dd, 1H, H-1′, J=4 and 22 Hz), 6.04 (dd, 1H, H-3′, J=2 and 16 Hz), 5.39 (ddd, 1H, H-2′, J=2, 4 and 50 Hz), 4.61-4.76 (m, 2H, 5′-CH₂), 1.58 (s, 3H, 4′-CH₃). 16α, TLC 2:1 hexane/EtOAc, R_{f} 0.52; MS m/z 511 (M+H)⁺; ¹H NMR (CDCl₃) 8.74 (s, 1H, H-2), 8.38 (s, 1H, H-8), 7.86-8.66 (m, 4H, ortho H's of benzoyl), 7.42-7.66 (m, 6H, para and meta H's of benzoyl), 6.51 (dd, 1H, H-1′, J=3.5 and 14 Hz), 6.24 (dt, 1H, H-2′, J=3 and 50 Hz), 6.0 (dd, 1H, H-3′, J=3 and 18 Hz), 4.46-4.72 (m, 2H, 5′-CH₂), 1.60 (s, 3H, 4′-CH₃).

### Example 16

9-(2-Deoxy-2-fluoro-4-C-methyl-β-D-arabinofuranosyl)adenine (17).^{[2]} Compound 16 (101 mg, 0.20 mmol) in a glass lined stainless steel bomb was diluted with ethanolic ammonia (100 mL, saturated at 5° C.). The sealed bomb was heated at 80° C. for 26 hours before the contents were evaporated. The residue was purified by multiple development on silica gel preparative TLC (Analtech GF, 10×20 cm, 10000 with 5:1 CHCl₃/MeOH+1% NH₄OH as solvent. Pure 17 (45 mg, 74%) was obtained as a white powder from acetone: m.p. 160-162° C.; TLC 5:1 CHCl₃/MeOH+1% NH₄OH, R_{f} 0.45; MS m/z 284 (M+H)⁺; UV λmax pH 1, 256 (15.0), pH 7, 259 (15.7), pH 13, 259 (16.3); ¹H NMR (DMSO-d₆) 8.30 (d, 1H, H-8, J=1 Hz), 8.14 (s, 1H, H-2), 7.32 (s, 2H, 6-NH₂), 6.43 (dd, 1H, H-1′, J=5 and 10 Hz), 5.94 (bs, 1H, 3′-OH), 5.35 (dt, 1H, H-2′, J=4 and 52 Hz), 5.22-5.30 (m, 1H, 5′-OH), 4.56 (dd, 1H, H-3′, J=4 and 20 Hz), 3.46-3.52 (m, 2H, 5′-CH₂), 1.16 (s, 3H, 4′-CH₃). Anal. Calcd. For C₁₁H₁₄FN₅O₃.0.40H₂O.0.30C₃H₆O: C, 46.42; H, 5.43; N, 22.75. Found: C, 46.44; H, 5.17; N, 22.83. 17α was prepared from 16α as described for 17. Pure 17α (49%) as a glass from acetone was ground to an off white powder: m.p. 185-187° C.; TLC 5:1 CHCl₃/MeOH+1% NH₄OH, R_{f} 0.51; MS m/z 284 (M+H)⁺; UV λmax pH 1, 257 (15.0), pH 7, 259 (15.5), pH 13, 259 (16.0); ¹H NMR (DMSO-d₆) 8.34 (s, 1H, H-8), 8.18 (s, 1H, H-2), 7.38 (s, 2H, 6-NH₂), 6.16 (dd, 1H, H-1′, J=5 and 10 Hz), 6.10 (bs, 1H, 3′-OH), 5.84 (dt, 1H, H-2′, J=4 and 52 Hz), 5.14 (t, 1H, 5′-OH, J=4 Hz), 4.49 (dd, 1H, H-3′, J=4 and 20 Hz), 3.32-3.40 (m, 2H, 5′-CH₂), 1.22 (s, 3H, 4′-CH₃). Anal. Calcd. For C₁₁H₁₄FN₅O₃.0.50H₂O.0.10C₃H₆O: C, 45.53; H, 5.28; N, 23.49. Found: C, 45.40; H, 5.02; N, 23.48.

### Example 17

2,6-Dichloro-9-(3,5-di-O-benzoyl-2-deoxy-2-fluoro-4-C-methyl-β-D-arabinofuranosyl)purine (18). Compound 18 was synthesized as described for 16 except using 2,6-dichloropurine. The anomeric product mixture was isolated by silica gel preparative TLC (developed twice in 3:1 hexane/EtOAc) as a white solid (64%, as 2:1β:α ratio by ¹H NMR). Pure anomers were obtained separately as white foams by preparative TLC with multiple developments in CHCl₃: 18, TLC 100:1 CHCl₃/MeOH, R_{f} 0.48; MS m/z 545 (M+H)⁺; ¹H NMR (CDCl₃) 8.40 (d, 1H, H-8, J=4 Hz), 8.10-8.16 (m, 4H, ortho H's of benzoyl), 7.46-7.72 (m, 6H, para and meta H's of benzoyl), 6.68 (dd, 1H, H-1′, J=3 and 20 Hz), 6.01 (dd, 1H, H-3′, J=1.5 and 16 Hz), 5.38 (ddd, 1H, H-2′, J=1.5, 4 and 50 Hz), 4.60-4.74 (m, 2H, 5′-CH₂), 1.56 (s, 3H, 4′-CH₃). 18α, TLC 100:1 CHCl₃/MeOH, R_{f} 0.42; MS m/z 545 (M+H)⁺; ¹H NMR (CDCl₃) 8.34 (s, 1H, H-8), 7.92-8.16 (m, 4H, ortho H's of benzoyl), 7.45-7.68 (m, 6H, para and meta H's of benzoyl), 6.48 (dd, 1H, H-1′, J=4 and 16 Hz), 6.12 (dt, 1H, H-2′, J=1.5 and 50 Hz), 6.01 (dd, 1H, H-3′, J=1.5 and 16 Hz), 4.48-4.72 (m, 2H, 5′-CH₂), 1.60 (s, 3H, 4′-CH₃).

### Example 18

2,6-Diazido-9-(3,5-di-O-benzoyl-2-deoxy-2-fluoro-4-C-methyl-β-D-arabinofuranosyl)purine (19). To a solution of 18 (108 mg, 0.20 mmol) in EtOH (5 mL) was added solid NaN₃ (30 mg, 0.46 mmol) and H₂O (0.5 mL). The mixture was placed in a 100° C. bath, refluxed 30 minutes, cooled, and evaporated. The residue was partitioned between CHCl₃ and H₂O. The aqueous layer was extracted twice with CHCl₃, and the combined organic layers were washed with H₂O, dried (MgSO₄), and evaporated to a syrup. Purification by silica gel preparative TLC (Analtech GF, 10×20 cm, 1000μ) in 2:1 hexane/EtOAc afforded 19 (105 mg, 95%) as a white solid that was used directly in the next step: TLC 99:1 CHCl₃/MeOH, R_{f} 0.65; MS m/z 559 (M+H)⁺; ¹HNMR (CDCl₃) 8.18 (d, 1H, H-8, J=4 Hz), 8.10-8.16 (m, 4H, ortho H's of benzoyl), 7.46-7.72 (m, 6H, para and meta H's of benzoyl), 6.62 (dd, 1H, H-1′, J=4 and 22 Hz), 6.0 (dd, 1H, H-3′, J=1.5 and 16 Hz), 5.34 (ddd, 1H, H-2′, J=1.5, 4 and 50 Hz), 4.90-4.70 (m, 2H, 5′-CH₂), 1.54 (s, 3H, 4′-CH₃). 19α was prepared from 18α as described for 19: TLC 99:1 CHCl₃/MeOH, R_{f} 0.58; MS m/z 559 (M+H)⁺; ¹H NMR (CDCl₃) 8.14 (s, 1H, H-8), 7.92-8.14 (m, 4H, ortho H's of benzoyl), 7.44-7.66 (m, 6H, para and meta H's of benzoyl), 6.41 (dd, 1H, H-1′, J=4 and 12 Hz), 6.16 (dt, 1H, H-2′, J=1.5 and 50 Hz), 6.01 (dd, 1H, H-3′, J=1.5 and 14 Hz), 4.48-4.68 (m, 2H, 5′-CH₂), 1.58 (s, 3H, 4′-CH₃).

### Example 19

2,6-Diamino-9-(3,5-di-O-benzoyl-2-deoxy-2-fluoro-4-C-methyl-β-D-arabinofuranosyl)purine (20). A solution of 19 (105 mg, 0.19 mmol) in 2:1 EtOH/DMAC (15 mL) was treated with 10% palladium on carbon (17 mg) and hydrogenated for 18 hours at room temperature and atmospheric pressure. The catalyst was removed by filtration and washed thoroughly with CHCl₃. The combined filtrate and washings were evaporated under vacuum to a syrup. Purification by silica gel preparative TLC (Analtech GF, 10×20 cm, 1000μ) with multiple development in 95:5 CHCl₃/MeOH+1% NH₄OH gave 20 (84 mg, 87%) as a white residue that was used directly in the next step: TLC 95:5 CHCl₃/MeOH+1% NH₄OH, R_{f} 0.43; MS m/z 507 (M+H)⁺; ¹H NMR (CDCl₃) 8.08-8.14 (m, 4H, ortho H's of benzoyl), 7.80 (d, 1H, H-8, J=3 Hz), 7.42-7.70 (m, 6H, para and meta H's of benzoyl), 6.48 (dd, 1H, H-1′, J=4 and 22 Hz), 6.01 (dd, 1H, H-3′, J=1.5 and 16 Hz), 5.30 (ddd, 1H, H-2′, J=1.5, 4 and 50 Hz), 5.38 (bs, 2H, 2-NH₂), 4.74 (bs, 2H, 6-NH₂), 4.60-4.68 (m, 2H, 5′-CH₂), 1.52 (s, 3H, 4′-CH₃). 20α was prepared from 19α as described for 20: TLC 95:5 CHCl₃/MeOH+1% NH₄OH, R_{f} 0.48; MS m/z 507 (M+H)⁺; ¹H NMR (CDCl₃) 8.12-8.16 (m, 4H, ortho H's of benzoyl), 7.78 (s, 1H, H-8), 7.44-7.96 (m, 6H, para and meta H's of benzoyl), 6.30 (dd, 1H, H-1′, J=3 and 16 Hz), 6.20 (dt, 1H, H-2′, J=1.5 and 16 Hz), 5.95 (dd, 1H, H-3′, J=2 and 14 Hz), 5.32 (bs, 2H, 2-NH₂), 4.66 (bs, 2H, 6-NH₂), 4.46-4.62 (m, 2H, 5′-CH₂), 1.56 (s, 3H, 4′-CH₃).

### Example 20

2,6-Diamino-9-(2-deoxy-2-fluoro-4-C-methyl-β-D-arabinofuranosyl)purine (21).^{[2]} To a solution of 20 (84 mg, 0.17 mmol) in MeOH (5 mL) at room temperature was added 0.5 N NaOCH₃ in MeOH (0.17 mL). The solution was stirred 3 hours, neutralized to pH 6 with glacial acetic acid, and evaporated. Purification by development on silica gel preparative TLC (Analtech GF, 10×20 cm, 500 μl) using 5:1 CHCl₃/MeOH+1% NH₄OH as solvent gave 21 (43 mg, 85%) as a white solid from acetone: m.p. 210-215° C.; TLC 5:1 CHCl₃/MeOH+1% NH₄OH, R_{f} 0.39; HPLC 99%, t_{R}=9.6 minutes, 20 minute linear gradient from 10-90% MeOH in 0.01M NH₄H₂PO₄ (pH 5.1); MS m/z 299 (M+H)⁺; HRMS m/z 299.12634 (M+H)⁺, Calcd. 299.12624 (M+H)⁺; UV λmax pH 1, 252 (11.5), 290 (9.9), pH 7, 255 (9.6), 280 (10.2), pH 13, 255 (9.8), 280 (10.5); ¹H NMR (DMSO-d₆) 7.84 (d, 1H, H-8, J=1 Hz), 6.74 (s, 2H, 2-NH₂), 6.20 (dd, 1H, H-1′, J=5 and 13 Hz), 5.82-5.92 (m, 3H, 6-NH₂ and 3′-OH), 5.24 (t, 1H, 5′-OH, J=4 Hz), 5.22 (dt, 1H, H-2′, J=4 and 52 Hz), 4.51 (dd, 1H, H-3′, J=4 and 20 Hz), 3.40-3.48 (m, 2H, 5′-CH₂), 1.14 (s, 3H, 4′-CH₃). Anal. Calcd. For C₁₁H₁₅FN₆O₃.0.40H₂O: C, 43.25; H, 5.21; N, 27.51. Found: C, 43.59; H, 5.09; N, 27.21. 21α was prepared from 20α as described for 21 except white solid was obtained from MeOH: m.p. 130-135° C.; TLC 5:1 CHCl₃/MeOH+1% NH₄OH, R_{f} 0.45; HPLC 99%, t_{R}=9.9 minutes, 20 minute linear gradient from 10-90% MeOH in 0.01M NH₄H₂PO₄ (pH 5.1); MS m/z 299 (M+H)⁺; HRMS m/z 299.12583 (M+H)⁺, Calcd. 299.12624 (M+H)⁺; UV λmax pH 1, 252 (12.4), 292 (10.5), pH 7, 255 (10.0), 280 (10.5), pH 13, 256 (9.8), 280 (10.5); ¹H NMR (DMSO-d₆) 7.92 (s, 1H, H-8), 6.76 (s, 2H, 2-NH₂), 6.78 (s, 1H, 3′-OH), 6.0 (dd, 1H, H-1′, J=4 and 16 Hz), 5.88 (s, 2H, 6-NH₂), 5.68 (dt, 1H, H-2′, J=4 and 52 Hz), 5.11 (t, 1H, 5′-OH, J=4 Hz), 4.44 (dd, 1H, H-3′, J=4 and 18 Hz), 3.32-3.38 (m, 2H, 5′-CH₂), 1.22 (s, 3H, 4′-CH₃). Anal. Calcd. For C₁₁H₁₅PN₆O3.1.5H₂O: C, 40.62; H, 5.58; N, 25.83. Found: C, 40.49; H, 5.26; N, 25.79.

### Example 21

9-(2-Deoxy-2-fluoro-4-C-methyl-β-D-arabinofuranosyl)guanine (22).^{[2]} A suspension of 21 (143 mg, 0.8 mmol) in H₂O (10 mL) was warmed to 70° C. to dissolve solid before being cooled to 30° C. Solid adenosine deaminase (22 mg, 33 units, type II: crude powder Sigma) was added, and the solution was stirred at room temperature. After 2.5 hours, the solution became cloudy, and the stirring was continued for 68 hours. The resulting milky mixture was filtered to remove crude 22 as a white solid (77 mg). The product containing filtrate was applied directly to a strong cation exchange resin in the H⁺ form (30 mL, AG 50W-X4, 100-200 mesh) equilibrated in H₂O. Elution with 0.25 N NH₄OH yielded 22 containing small amounts of UV active impurities. These impurities were removed by preparative TLC on silica gel (Analtech GF, 10×20 cm, 1000μ) using 9:2 MeCN/1N NH₄OH as eluent to provide more crude 22 (66 mg) as a white solid. Both solids were combined in hot water, and the solution was diluted to 50 mL. This solution at room temperature was applied to a XAD-4 resin column (100-200 mesh, 1×8.5 cm) equilibrated in H₂O. Elution with H₂O was continued followed by 9:1 H₂O/MeOH, when 22 appeared in the eluate. The pooled product containing fractions were evaporated and the residue was triturated with EtOH (25 mL) to give pure 22 (96 mg, 67%) as a white solid: m.p. 270° C. (dec.); TLC 9:2 MeOH/1N NH₄OH, R_{f} 0.55; HPLC 100%, t_{R}=8.3 minutes, 85:15 NH₄H₂PO₄ (0.01M, pH 5.1)/MeOH; MS m/z 300 (M+H)⁺; HRMS m/z 322.09177 (M+Na)⁺, Calcd. 322.09220 (M+Na)⁺; UV λmax pH 1, 256 (13.1), 280 (sh), pH 7, 251 (14.5), 276 (sh), pH 13, 263 (12.1); ¹H NMR (DMSO-d₆) 10.66 (s, 1H, 3-NH), 7.88 (d, 1H, H-8, J=1 Hz), 6.50 (s, 2H, 2-NH₂), 6.14 (dd, 1H, H-1′, J=5 and 13 Hz), 5.90 (d, 1H, 3′-OH, J=5 Hz), 5.23 (dt, 1H, H-2′, J=4 and 52 Hz), 5.17 (t, 1H, 5′-OH, J=4 Hz), 4.45 (dt, 1H, H-3′, J=4 and 18 Hz), 3.32-3.48 (m, 2H, 5′-CH₂), 1.16 (s, 3H, 4′-CH₃). Anal. Calcd. For C₁₁H₁₄FN₅O₄: C, 44.15; H, 4.72; N, 23.40. Found: C, 43.93; H, 4.69; N, 23.19.

### Example 22

2-Chloro-6-methoxy-9-(2-deoxy-2-fluoro-4-C-methyl-β-D-arabinofuranosyl)purine (23). To a solution of 18 (76 mg, 0.14 mmol) in anhydrous MeOH (5 mL) at room temperature was added dropwise 0.5 N NaOCH₃ in MeOH (280 μl). The solution was stirred for 3 hours neutralized to pH 6 with glacial acetic acid, and evaporated. The residue was purified by preparative TLC on silica gel using 9:1 CHCl₃/MeOH as solvent. Pure 23 (43 mg, 93%) was obtained as a clear glass and was used directly in the next step: TLC 9:1 CHCl₃/MeOH, R_{f} 0.48; MS m/z 333 (M+H)⁺; ¹H NMR (DMSO-d₆) 8.64 (d, 1H, H-8, J=1 Hz), 6.47 (dd, 1H, H-1′, J=5 and 10 Hz), 5.98 (bs, 1H, 3′-OH), 5.43 (dt, 1H, H-2′, J=4 and 52 Hz), 5.30 (t, 1H, 5′-OH, J=4 Hz), 4.54 (dd, 1H, H-3′, J=5 and 20 Hz), 4.12 (s, 3H, 6-OCH₃), 3.48-3.52 (m, 2H, 5′-CH₂), 1.14 (s, 3H, 4′-CH₃). 23α was prepared from 18α as described for 23. Pure 23α (96%) was obtained as a glass: TLC 9:1 CHCl₃/MeOH, R_{f} 0.48; MS m/z 333 (M+H)⁺; ¹H NMR (DMSO-d₆) 8.62 (d, 1H, H-8, J=1 Hz), 6.20 (dd, 1H, H-1′, J=5 and 14 Hz), 6.16 (bs, 1H, 3′-OH), 5.76 (dt, 1H, H-2′, J=4 and 52 Hz), 5.24 (bt, 1H, 5′-OH), 4.54 (dd, 1H, H-3′, J=5 and 20 Hz), 4.12 (s, 3H, 6-OCH₃), 3.34-3.38 (m, 2H, 5′-CH₂), 1.24 (s, 3H, 4′-CH₃).

### Example 23

2-Chloro-9-(2-deoxy-2-fluoro-4-C-methyl-β-D-arabinofuranosyl)adenine (24). A solution of 23 (88 mg, 0.27 mmol) in 60 mL of ethanolic ammonia (saturated at 5° C.) in a glass-lined stainless steel bomb was heated at 80° C. for 21 hours and evaporated. The residue was purified by silica gel preparative TLC (Analtech GF, 10×20 cm, 1000μ) with two developments in 5:1 CHCl₃/MeOH+1% NH₄OH. Pure 24 (71 mg, 84%) as a white foam from acetone was ground to a white powder: m.p. 220-222° C.; TLC 9:1 CHCl₃/MeOH+1% NH₄OH, R_{f} 0.29; HPLC 97%, t_{R}=14 minutes, 4:1 NH₄H₂PO₄ (0.01M, pH 2.7)/MeOH; MS m/z 318 (M+H)⁺; UV λmax pH 1, 264 (14.7), pH 7, 264 (15.4), pH 13, 264 (15.8); ¹H NMR (DMSO-d₆) 8.44 (d, 1H, H-8, J=1 Hz), 7.86 (s, 2H, 6-NH₂), 6.35 (dd, 1H, H-1′, J=5 and 10 Hz), 5.94 (d, 1H, 3′-OH, J=6 Hz), 5.37 (dt, 1H, H-2′, J=4 and 52 Hz), 5.25 (t, 1H, 5′-OH, J=4 Hz), 4.53 (dd, 1H, H-3′, J=4 and 20 Hz), 3.48-3.50 (m, 2H, 5′-CH₂), 1.14 (s, 3H, 4′-CH₃). Anal. Calcd. For C₁₁H₁₃ClFN₅O₃: C, 41.59; H, 4.12; N, 22.04. Found: C, 41.55; H, 4.12; N, 22.06. 24α was prepared from 23α as described for 24. Pure 24α (75%) was recovered from acetone as a white solid: m.p., dual 105° C. and 205° C.; TLC 9:1 CHCl₃/MeOH+1% NH₄OH, R_{f} 0.35; HPLC 98%, t_{R}=13 minutes, 4:1 NH₄H₂PO₄ (0.01M, pH 2.7)/MeOH; MS m/z 318 (M+H)⁺; UV λmax pH 1, 264 (15.1), pH 7, 264 (16.1), pH 13, 264 (15.9); ¹H NMR (DMSO-d₆) 8.38 (d, 1H, H-8, J=1 Hz), 7.90 (s, 2H, 6-NH2), 6.09 (dd, 1H, H-1′, J=5 and 14 Hz), 6.02 (bs, 1H, 3′-OH), 5.74 (dt, 1H, H-2′, J=4 and 52 Hz), 5.16 (t, 1H, 5′-OH, J=4 Hz), 4.50 (dd, 1H, H-3′, J=4 and 20 Hz), 3.32-3.38 (m, 2H, CH₂), 1.24 (s, 3H, 4′-CH₃). Anal. Calcd. For C₁₁H₁₃ClFN₅O₃.0.65H₂O.0.10C₃H₆O: C, 40.49; H, 4.48; N, 20.89. Found: C, 40.43; H, 4.35; N, 20.72.

In keeping with the present disclosure, the compounds of the present disclosure can be used alone or in appropriate association, and also may be used in combination with pharmaceutically acceptable carriers and other pharmaceutically active compounds such as various cancer treatment drugs and/or along with radiation. The active agent may be present in the pharmaceutical composition in any suitable quantity.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art.

The compounds of the present disclosure alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example. dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-does or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4^{th} ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of exogenously administering a compound of the present disclosure to an animal are available, and, although more than one route can be used to administer a particular compound, a particular route can provide a more immediate and more effective reaction than another route.

The present disclosure further provides a method of cancer in a mammal, especially humans. The method comprises administering an effective treatment amount of a compound as disclosed above to the mammal.

As regards these applications, the present method includes the administration to an animal, particularly a mammal, and more particularly a human, of a therapeutically effective amount of the compound effective in the inhibition of neoplasia and tumor growth and treating malignant disease including metastases.

The disclosed compounds and compositions can be administered to treat a number of cancers, including leukemias and lymphomas such as acute lymphocytic leukemia, acute nonlymphocytic leukemias, chronic lymphocytic leukemia, chronic myelogenous leukemia, Hodgkin's Disease, non-Hodgkin's lymphomas, and multiple myeloma, childhood solid tumors such as brain tumors, neuroblastoma, retinoblastoma, Wilms Tumor, bone tumors, and soft-tissue sarcomas, common solid tumors of adults such as lung cancer, breast cancer, prostate cancer, urinary cancers, uterine cancers, oral cancers, pancreatic cancer, melanoma and other skin cancers, stomach cancer, ovarian cancer, brain tumors, liver cancer, laryngeal cancer, thyroid cancer, esophageal cancer, and testicular cancer.

The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the cancer.

A suitable dose is that which will result in a concentration of the active agent in tumor tissue which is known to affect the desired response. The preferred dosage is the amount which results in maximum inhibition of cancer, without unmanageable side effects.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

The method disclosed comprises further administering of chemotherapeutic agent other than the derivatives of the present invention. Any suitable chemotherapeutic agent can be employed for this purpose. The chemotherapeutic agent is typically selected from the group consisting of alkylating agents, antimetabolites, natural products, anti-inflammatory agents, hormonal agents, molecular targeted drugs, anti-angiogenic drugs, and miscellaneous agents.

Examples of alkylating chemotherapeutic agents include carmustine, chlorambucil, cisplatin, lomustine, cyclophosphamide, melphalan, mechlorethamine, procarbazine, thiotepa, uracil mustard, triethylenemelamine, busulfan, pipobroman, streptozocin, ifosfamide, dacarbazine, carboplatin, and hexamethylmelamine.

Examples of chemotherapeutic agents that are antimetabolites include cytosine arabinoside fluorouracil, gemcitabine, mercaptopurine, methotrexate, thioguanine, floxuridine, fludarabine, and cladribine.

Examples of chemotherapeutic agents that are natural products include actinomycin D, bleomycin, camptothecins, daunomycin, doxorubicin, etoposide, mitomycin C, paclitaxel, taxoteredocetaxel, tenisposide, vincristine, vinblastine, vinorelbine, idarubicin, mitoxantrone, mithramycin and deoxycoformycin.

Examples of hormonal agents include antiestrogen receptor antagonists such as tamoxifen and fluvestrant, aromatase inhibitors such as anastrozole, androgen receptor antagonists such as cyproterone and flutamine, as well as gonadotropin release hormone agonists such as leuprolide. Examples of anti-inflammatory drugs include adrenocorticoids such as prednisone, and nonsteroidal anti-inflammatory drugs such as sulindac or celecoxib. Examples of molecular targeted drugs include monoclonal antibodies such as rituximab, cetuximab, trastuzumab and small molecules such as imatinib, erlotinib, ortizumib. Examples of anti-angiogenic drugs include thalidomide and bevacizimab. Examples of the aforesaid miscellaneous chemotherapeutic agents include mitotane, arsenic trioxide, tretinoin, thalidomide, levamisole, L-asparaginase and hydroxyurea.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference.

### REFERENCES

1. Shortnacy-Fowler, A. T., Tiwari, K. N., Montgomery, J. A. and Secrist, J. A. III. Synthesis and Biological Activity of 4′-C-hydroxymethyl-2′-fluoro-D-arabinofuranosylpurine Nucleosides. Nucleosides Nucleotides & Nucleic Acids, 2001, 20, 1583-1598.
2. Chang, J., Bao, X., Wang, Q., Guo, X., Wang, W. and Qi, X. Preparation of 2′-fluoro-4′-Substituted Nucleoside Analogs as Antiviral Agents. Chinese Patent Application #CN 2007-10137548, 20070807.
3. Mandal, S. B. and Achari, B. Stereospecific C-β-Glycosidation and Synthesis of 4,7-Anhydro-5,6-Isopropylidene-4(S),5(S),6(R),7(R)-Tetrahydroxyoxocan-2-One. Synthetic Communications, 1993, 23(9), 1239-1244.
4. Gunic, E., Girardet, J.-L., Pietrzkowski, Z., Esler, C. and Wang, G. Synthesis and Cytotoxicity of 4′-C- and 5′-C-Substituted Toyocamycins. Bioorganic & Medicinal Chemistry, 2001, 9, 163-170.
5. Ohrui, H., Kohgo, S., Kitano, K., Sakata, S., Kodama, E., Yoshimura, K., Matsuoka, M., Shigeta, S, and Mitsuya, H. Syntheses of 4′-C-Ethynyl-β-D-arabino- and 4′-C-Ethynyl-2′-deoxy-β-D-ribo-pentofuranosylpyrimidines and purines and Evaluation of Their Anti-HIV Activity. J. Med. Chem., 2000, 43, 4516-4525.
6. Siddiqui, M. A., Driscoll, J. S., Marquez, V. E., Roth, J. S., Shirasaka, T., Mitsuya, H., Barchi, J. J. Jr. and Kelley, J. A. Chemistry and Anti-HIV Properties of 2′-Fluoro-2′,3′-dideoxyarabinofuranosylpyrimidines. J. Med. Chem., 1992, 35, 2195-2201.
7. Kazimierczuk, Z., Cottam, H. B., Revankar, G. R. and Robins, R. K. Synthesis of 2′-Deoxytubercidin, 2′-Deoxyadenosine, and Related 2′-Deoxynucleosides via a Novel Direct Stereospecific Sodium Salt Glycosylation Procedure. J. Am. Chem. Soc., 1984, 106, 6379-6382.
8. Secrist, J. A. III, Tiwari, K. N., Shortnacy-Fowler, A. T., Messini, L., Riordan, J. M. and Montgomery, J. A. Synthesis and Biological Activity of Certain 4′-Thio-D-arabinofuranosylpurine Nucleosides. J. Med. Chem., 1998, 41, 3865-3871.

What is claimed is:

1. A compound represented by the formula: 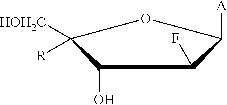
wherein A is 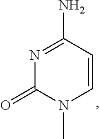
and
wherein R is alkyl; and pharmaceutically acceptable salts thereof.

2. The compound of claim 1 wherein R is an alkyl containing 1 to 4 carbon atoms.

3. The compound of claim 1 wherein R is methyl.

4. A pharmaceutical composition comprising a compound of claim 1 and a pharmaceutically acceptable carrier.

5. A method of treating cancer in a mammal comprising administering to the mammal an effective treatment amount of a compound represented by the formula: 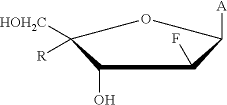
wherein R is alkyl,
wherein A is selected from the group consisting of 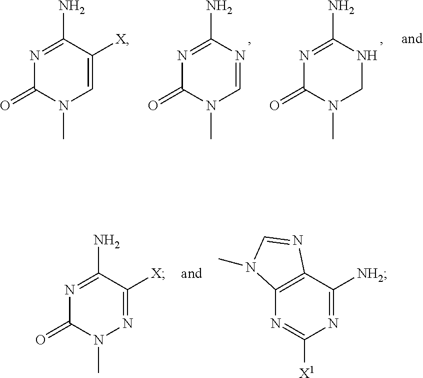
and wherein X is selected from the group consisting of hydrogen, halo, alkoxy, alkyl, haloalkyl, alkenyl, haloalkenyl, alkynyl, amino, monoalkylamino, dialkylamino, cyano and nitro; and X¹ is selected from the group consisting of hydrogen, halo, alkyl, alkenyl, alkynyl, amino, monoalkylamino, and dialkylamino; and pharmaceutically acceptable salts thereof.

6. The method according to claim 5, wherein R is an alkyl containing 1 to 4 carbons.

7. The method according to claim 5, wherein R is methyl.

8. The method according to claim 5, wherein said compound is 9-(2-Deoxy-2-fluoro-4-C-methyl-β-D-arabinofuranosyl)adenine.

9. The method according to claim 5, wherein said compound is 2,6-Diamino-9-(2-deoxy-2-fluoro-4-C-methyl-β-D-arabinofuranosyl)purine.

10. The method according to claim 5, wherein said compound is 2-Chloro-9-(2-deoxy-2-fluoro-4-C-methyl-β-D-arabinofuranosyl)adenine.

11. A method of treating cancer in a mammal comprising administering to the mammal an effective treatment amount of a compound according to claim 1.

12. The method according to claim 11, wherein R is an alkyl containing 1 to 4 carbons.

13. The method according to claim 11, wherein R is methyl.
